(19) United States
(12) Patent Application Publication
(10) Pub. No.: US 2016/0237112 A1
(43) Pub. Date: Aug. 18, 2016

(54) PURIFICATION OF ORGANIC COMPOUNDS BY SURFACTANT MEDIATED PREPARATIVE HPLC

(71) Applicant: DAVULURI, Ramamohan Rao, Hyderabad (IN)

(72) Inventor: Mohammed Khalid ANWER, HYDERABAD (IN)

(21) Appl. No.: 15/022,983

(22) PCT Filed: Sep. 18, 2014
(86) PCT No.: PCT/IN2014/000607

§ 371 Date: Mar. 18, 2016

(30) Foreign Application Priority Data
Sep. 20, 2013 (IN) 4264/CHE/2013

(51) Int. Cl. C07K 1/20; B01D 15/32; C07K 7/23

(52) U.S. Cl. CPC C07K 1/20; C07K 7/23; B01D 15/325

(57) ABSTRACT

A sample is provided that can be purified by preparative reversed phase high performance liquid chromatography (Prep-RP-HPLC) in a single run in spite of recent advances in the production of reversed phase derivatized silica stationary supports: (1) The traditional approach is to use a bigger column (greater amount of stationary phase); and (2) Use displacement chromatography which (while labor intensive to develop) uses the stationary phase more effectively. This disclosure describes a unique Prep-RP-HPLC technique that uses a C-18/C-8 derivatized silica coated with a surfactant such as Triton X-100 to result in 7 to 10 fold increase in sample loading (of the crude mixture of organic compounds including synthetic crude peptides) in contrast to the conventional Prep-RP-HPLC technique. This increase in sample loading capacity and output is due to the additional surrogate stationary phase characteristic of the C-18/C8 adsorbed (bound) surfactant.

### CROSS-REFERENCE TO RELATED APPLICATIONS

This application claims priority to PCT Application No. PCT/IN2014/000607, having a filing date of Sep. 18, 2014, based on IN 4264/CHE/2013, having a filing date of Sep. 20, 2013, the entire contents of which are hereby incorporated by reference.

### FIELD OF TECHNOLOGY

The following relates to purification of organic compounds. More particularly, the following relates to a novel method of purification of organic compounds including peptides using Preparative Reversed Phase High Performance Liquid Chromatography (Prep-RP-HPLC) technique which has 7 to 10 times greater sample loading capacity, and output compared to the traditional Prep-RP-HPLC for the purification of organic compounds including peptides using surfactants as surrogate stationary phases (SSPs)/additional stationary phases (ASPs). The increased sample loading capacity is due to the adsorbed surfactant on the C-18/C8 chains acting as the additional stationary phase (ASP).

### BACKGROUND

Reversed Phase High Performance Liquid Chromatography (RP-HPLC) is used ubiquitously in academic institutions, forensic laboratories, fine chemicals, and pharmaceutical industries etc. for the analysis, characterization, separation, purification and/or isolation of small organic molecules, natural products, and biologically active molecules such as polypeptides, proteins, and nucleotides. In the pharmaceutical industry, analytical RP-HPLC is used for the release and characterization of raw materials, intermediates, and active pharmaceutical ingredients (APIs). In contrast, preparative chromatography is used to purify sufficient quantities of a substance for further use. While the primary goal of analytical RP-HPLC is identification and quantitation of analytes, the primary objective of Prep-RP-HPLC in the pharmaceutical and fine chemicals industries is the commercial production of APIs including Peptide APIs, and most other complex APIs that are not amenable to crystallization, and fine chemicals.

Prep-RP-HPLC in the elution mode is the most widely practiced and preferred mode for purifying crude peptide mixtures, and other small complex organic molecules due to the ease of elution mode operation. In the elution preparative chromatography the crude mixture of compounds to be purified is dissolved in a suitable solvent {for example, 0.1% trifluoroacetic acid (TFA) in water, Buffer A} and bound to the C-18/C-8 derivatized silica stationary phase support. As the mobile phase (0.1% TFA in 50% to 100% acetonitrile, Buffer B) gradient (usually a linear gradient of A to B) is run, equilibrium is established between the mobile phase and stationary phase. Depending on their affinity for the stationary phase, the various sample species traverse along the column at speeds reflecting their relative affinity for the stationary phase. The weakly bound species elute first followed by the stronger binders. In summary gradual increasing of the concentration of the organic buffer component results in desorption, and resolution of the components of the mixture.

The elution Prep-RP-HPLC mode is limited in terms of the quantity of sample that can be purified in a single run by several factors including the resolution between the desired product and its closest eluting related substance, the capacity factor, and the number of theoretical plates of the preparative column etc. Donald A. Wellings has elegantly described many of these aspects in his book “A Practical Handbook of Preparative HPLC, Elsevier (2006)”. The typical loading capacity of synthetic peptides is in the range of 1 to 2 mgs per ml of packed column volume (viz., 0.1% to 0.2% with respect to total column volume).

The earlier Prep-RP-HPLC stationary supports were irregular silica particles that were derivatized with C-18 or C-8 chains, and they suffered from high back pressure. The high back pressure limited their use with respect to the quantity that could be purified in a single run, and to relatively smaller diameter columns. Recent advances in Prep-RP-HPLC have focused on producing spherical silica and development of new bonding chemistries to furnish stationary supports that have improved stability and selectivity. The commercial manufacture of spherical silica that has been derivatized by C-18, C-8, and other ligands has overcome these challenges and has extended the utility of preparative HPLC vastly. The technological advances in process HPLC instrumentation, and the bonded silica supports have made possible commercial production of complex peptides such as Fuzeon®, a 36-amino acid peptide, in hundreds of kilos quantities. Unfortunately, these large scale HPLC instruments and the associated column hardware are very costly and restrict the affordability of the methods. Also, none of these improvements have addressed the loading capacity of a given column nor have they resulted in significant increase in the amount of purified product (output/mL of the packed column).

As eluded earlier, in spite of these aforementioned advances, there are only two ways to increase the amount of sample that can be purified by Prep-RP-HPLC in a single run: (1) The traditional approach is to use a bigger column (greater amount of stationary phase); and (2) Use displacement chromatography which (while laborious to develop) uses the stationary phase more effectively.

Displacement chromatography utilizes a mobile phase displacer solution which has higher affinity for the stationary phase material than do the sample components. The key operational feature which distinguishes displacement chromatography from elution chromatography is the use of a displacer molecule. In elution chromatography, the eluent usually has a lower affinity for the stationary phase than do any of the components in the mixture to be separated, whereas in displacement chromatography, the eluent, which is the displacer, has a higher affinity. The displacement is best suited for ion exchange mode, and has found numerous recent applications. The U.S. Pat. No. 6,239,262 discloses low molecular weight displacers for protein purification in the hydrophobic interaction and reverse phase chromatography modes.

Embodiments of the invention achieve higher output by making use of the SSP. The SSP and the displacement chromatography act synergistically to increase the output of the preparative chromatography.

Inventor's PCT application WO 2014/118797 A1 describes a unique Prep-RP-HPLC technique that achieves a 7 to 10 fold increase in sample loading (of the crude organic mixture of compounds including synthetic crude peptides) in contrast to the conventional Prep-RP-HPLC technique. This increase in output compared to conventional Prep-RP-HPLC technique is due to the additional surrogate stationary phase characteristic of the C-18/C-8 adsorbed (bound) quaternary ammonium salt. The quaternary ammonium salt is bound to the C-18/C-8 chains of the stationary phase via Van der Waals forces (hydrophobic interactions) and ionic interactions with the residual silanols of the stationary phase.

Embodiments of the invention describes the use of C-18/C-8 derivatized silica coated with neutral surfactants such as Triton X-100 as the ASP (please see FIG. 3). Embodiments of the present invention describes a scalable separation process for peptides using Prep-RP-HPLC and neutral surfactants as SSP/ASP. Embodiments of the invention are a simple, cost effective and scalable separation process for peptides.

### SUMMARY

An aspect relates to a novel method of purification of organic compounds including peptides using Preparative Reversed Phase High Performance Liquid Chromatography (Prep-RP-HPLC) technique.

Another aspect is to provide a method or purification of organic compounds including peptides which has 7 to 10 times greater sample loading capacity, and output compared to the traditional Prep-RP-HPLC technique.

A further aspect is to provide such method using surfactants as surrogate stationary phases (SSPs)/additional stationary phases (ASPs).

Accordingly, in one embodiment, the embodiments of the invention provides a method of purification of organic compounds including peptides with increased sample loading capacity of reverse phase column in Preparative Reversed Phase High Performance Liquid Chromatography (Prep-RP-HPLC) using a surrogate stationary phase/additional stationary phase in conjunction with a C-18/C-8 derivatized silica stationary phase. The preparative loading capacity of C-18/C-8 reverse phase column is increased by coating/binding the C-18/C-8 reverse phase column with a surrogate stationary phase/additional stationary phase, wherein the surrogate stationary phase/additional stationary phase is a neutral surfactant or pegylated surfactant.

The surrogate stationary phase/additional stationary phase surfactants may be selected from alkyl glycoside, bile acids, glucamides and poly-oxyethylenes, wherein the poly-oxyethylenes are selected from Triton X-100, Tween-80 and Brij-35, preferably Triton X-100. The alkyl glycosides are selected from the compounds have the formula of—

R—O—(CH₂)ₓ—CH₃,

### Wherein,

### When R=Glucose When R=Maltose

X=8, n-nonyl-β-D-glucopyranoside x=11, dodecyl-β-D-maltoside
x=7, n-octyl-β-D-glucopyranoside x=9, dodecyl-β-D-maltoside
X=6, n-heptyl-β-D-glucopyranoside x=9, decyl-β-D-maltoside
x=5, n-hexyl-β-D-glucopyranoside.

The bile acids are selected from the compounds having formula:

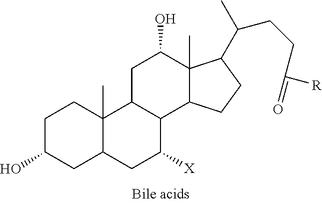

wherein,
X═H, R═ONa, Sodium deoxycholate
X═H, R═NHCH₂CH₂SO₃Na, Sodium taurodeoxycholate
X═H, R═NHCH₂CH₂CO₂Na, Sodium glycodeoxycholate
X═OH, R═ONa, Sodium cholate
X═OH, R═NHCH₂CH₂SO₃Na, Sodium taurocholate
X═OH, R═NHCH₂CH₂CO₂Na, Sodium glycocholate

The glucamides are selected from the compounds having formula:

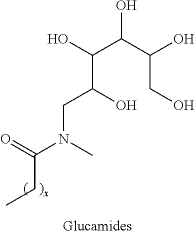

### Wherein,

### X=8, MEGA-10

### X=7, MEGA-9,

### X=6, MEGA-8

Or, compound of the formula:

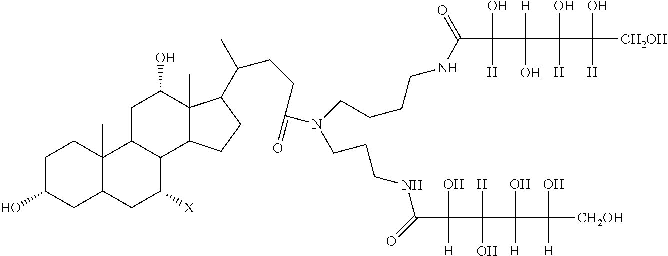

### Wherein,

### X═H, Deoxy Big CHAP

### X═OH, Big CHAP

In another embodiment, the invention provides a method of purifying a multi-component sample of organic compounds including peptides by Preparative Reversed Phase High Performance Liquid Chromatography (Prep-RP-HPLC) comprising the steps of:
(a) configuring a chromatographic system having a hydrophobic stationary phase;
(b) saturating the chromatographic stationary phase with surrogate stationary phase/additional stationary phase surfactants selected from alkyl glycoside, bile acids, glucamides and poly-oxyethylenes;
(c) washing the column to remove excess un-bound surfactant employing a mixture of organic solvents and water;
(d) equilibrating the column with the starting mobile phase;
(e) applying a multicomponent sample to one end of the chromatographic bed comprising stationary phase coated with the surfactants; and
(f) eluting the multicomponent sample using a linear gradient of buffers A & B, wherein the buffer A is 0.1 mM Cetyltrimethylammonium bromide and 0.1 mM sodium bicarbonate in water and buffer B is 0.1 mM Cetyltrimethylammonium bromide and 0.1 mM sodium bicarbonate in 50% aqueous acetonitrile;
(g) eluting the multicomponent sample using a linear gradient of buffers A & B, wherein the buffer A is 0.1% phosphoric acid in water and buffer B is 0.1% phosphoric acid in 50% aqueous acetonitrile; or
(h) eluting the multicomponent sample using a linear gradient of buffers A & B, wherein the buffer A is 1% phosphoric acid in water and buffer B is 1% phosphoric acid in 50% aqueous acetonitrile; or
(i) eluting the multicomponent sample using a linear gradient of buffers A & B, wherein the buffer A is 25 mM to 150 mM triethylammonium phosphate (pH 3) in water and buffer B is 25 mM to 150 mM triethylammonium phosphate (pH 3) in 50% aqueous acetonitrile; and
(j) recovering the desired component of the sample.

The hydrophobic stationary phase in step (a) is C-8 or C-18 alkyl chain derivatized silica and the surfactants in step (b) is selected from Triton X-100, Tween-80, and Brij-35.

The washing of the column in step (c) to remove the un-bound surfactant comprises washing the column with aqueous acetonitrile more preferably 90% aqueous acetonitrile containing 0.1% trifluoroacetic acid and the equilibration comprises equilibrating the column with the starting mobile phase more preferably 0.1% to 1% aqueous phosphoric acid, 0.1% TFA in water, and 25 to 150 mM triethylammonium phosphate in water.

In another embodiment, the invention provides a method of purification of organic compounds including peptides with increased sample loading capacity of reverse phase column in Preparative Reversed Phase High Performance Liquid Chromatography (Prep-RP-HPLC) using a PEG based detergent/surfactant which has the following structure as ASP/SSP in conjunction with C-18/C-8 derivatized silica or other supports as the stationary phase:

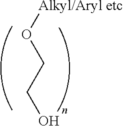

wherein alkyl/Aryl etc. are selected independently from the group comprising straight or branched alkyl, cyclic hydrocarbons, aromatic group, alkyl substituted aromatic group, aryl substituted alkyl groups; And “n” is the number of ethylene-oxide residues from 1 to 20 preferably 6 to 12, more preferably 9 to 10.

The increase in sample loading capacity of embodiments of the invention occurs when the surrogate stationary phase bound to C-18 derivatized silica is mobile (as observed with lower carbon based surfactants where concurrent binding and leaching from the stationary phase are seen), and also when it is tightly to permanently bound to the C-18/C-8 reversed stationary phase, wherein the C-18/C-8 reversed stationary phase is selected from Triton X-100, Brij-35 and Tween-80.

In another embodiment, the invention also provides a method for removing the surrogate stationary phase/additional stationary phase coating from the C-18/C-8 derivatized silica support by washing the column with a buffer capable of H-bonding with the residual silanols and having sufficient concentration of organic modifier, wherein the organic modifier is 0.25M to 0.5M ammonium acetate in 50% to 90% aqueous acetonitrile.

Embodiments of the invention have various industrial advantages such as limited use of solvents, reduced waste disposal, ease of operation and lower scale of the equipments.

### BRIEF DESCRIPTION

Some of the embodiments will be described in detail, with reference to the following figures, wherein like designations denote like members, wherein:

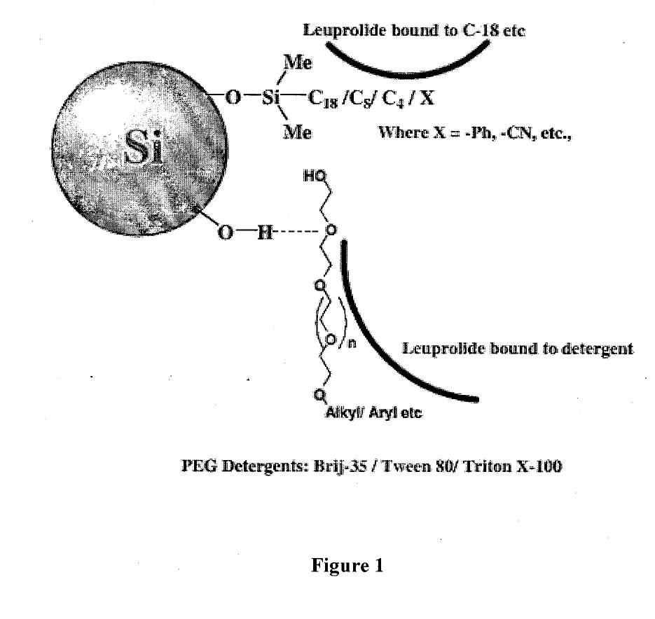

FIG. 1: Leuprolide bound to C-18 and PEG-based Surrogate Stationary Phases.

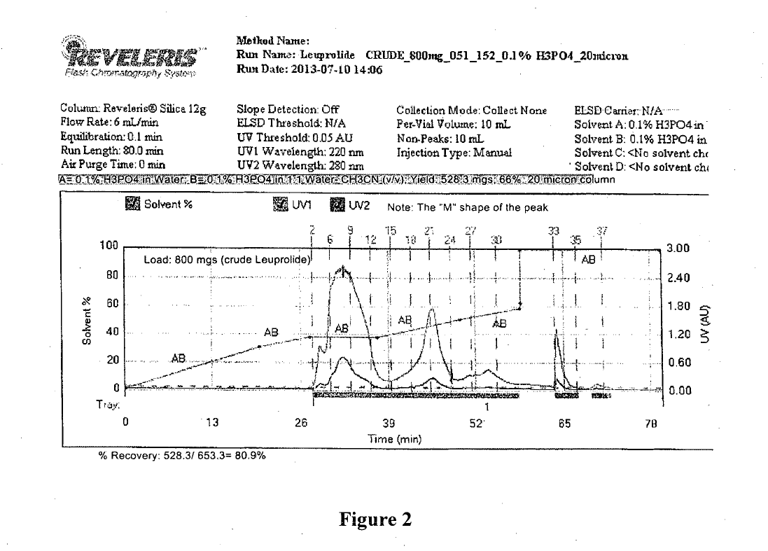

FIG. 2: Prep-RP-HPLC profile of 800 mgs net Leuprolide loaded on to a 12 g C-18 Reveleris® (flash chromotography system) Column Coated with Triton X-100. (Fractions 8 through 24 contained Leuprolide that was >97.9% pure by analytical RP-HPLC using a modified EP method for Leuprolide)

### DETAILED DESCRIPTION

Table 1 describes the loading capacity of various chromatography techniques (entries 1 to 4). Entries 5 and 6 pertain to the loading capacity when the C-18/C-8 support is coated with the surrogate stationary phase.

TABLE 1
Loading Capacity of Various Chromatographic Techniques

| | | | | | | | | | Relative |
|---|---|---|---|---|---|---|---|---|---|
| | | | | | | | | % | Loading |
| | | | | | | | | Loading | Capacity |
| | | | Total | | | Amount | | wrt | (Standard |
| | | | Column | ID | Type of | of | Amount | Total | RP- |
| | Source/ | Type of | Volume | (cm) × L | Stationary | Stationary | of crude | Column | HPLC = |
| Entry # | Operator | Chromatography | (mL) | (cm) | Phase | Phase | purified | Volume | 1) |
| #1 | Donald | RP- | 4420 | 15 × 25 | C-18 | 3.5 Kg | 40 g of | 0.9% | 1.0 |
| | A. | HPLC | | | | | Crude | | |
| | Wellings | | | | | | Peptide | | |
| | (A | | | | | | | | |
| | Practical | | | | | | | | |
| | Handbok | | | | | | | | |
| | of | | | | | | | | |
| | Preparative | | | | | | | | |
| | HPLC, | | | | | | | | |
| | Elsevier | | | | | | | | |
| | (2006) | | | | | | | | |
| #2 | Donald | Self | 347 | 4.2 × 25 | PLRP-S | ? | 6.2 g of | 1.8% | 2.0 |
| | A. | displacement- | | | | | a 25-AA | | |
| | Wellings- | RP- | | | | | Peptide | | |
| | As | HPLC | | | | | | | |
| | Above | | | | | | | | |
| #3 | Donald | Enantiomers | 1964 | 10 × 25 | Chiralpak | ? | 120 g (6 | 6.1% | 6.8 |
| | A. | separation | | | AD | | boxcar | | |
| | Wellings- | | | | | | injections | | |
| | As | | | | | | of 20 g | | |
| | Above | | | | | | each | | |
| | | | | | | | injection) | | |
| #4 | Donald | NP- | 1964 | 100 × 25 | Silica | 155 Kg | 15,500 g | 7.9% | 8.7 |
| | A. | HPLC | 29 | | | | | | |
| | Wellings- | (Normal | | | | | | | |
| | As | Phase) | | | | | | | |
| | Above | | | | | | | | |
| #5 | Neuland | RP- | 19.6 | 1 × 25 | Discovery | 11 g | 1.4 g | 7.1% | 7.9 |
| | Health | HPLC | | | Wide | | Crude | | |
| | Sciences | | | | Pore C- | | Leuprolide | | |
| | Private | | | | 18, 5μ, | | | | |
| | Limited | | | | 100 Å | | | | |
| | | | | | pores | | | | |
| #6 | Neuland | RP- | 14.1 | 1.9 × 5 | Waters | 11 g | 1.4 g | 9.9% | 11.0 |
| | Health | HPLC | | | Symmetry | | Crude | | |
| | Sciences | | | | C-8, | | Leuprolide | | |
| | Private | | | | 5μ, 100 Å | | | | |
| | Limited | | | | pores | | | | |

The typical loading capacity of a reversed phase column is about 0.90% with respect to the volume of the packed column (Table 1, Entry #1). The sample loading capacity is greater in displacement chromatography because of the better utilization of the available stationary phase (PLRP-S, Polystyrene Column) for resolving the components of the crude peptide mixture, and in this instance was about 2% with respect to the total column volume (Table 1, Entry 2). WO 2013/052539 describes the use of displacement chromatography (DC) purification of peptides such as Angiotensin etc. The DC of angiotensin used a Waters Xbridge BEH130 {C-18, 5 micron, 135 Angstroms (Å), 0.46 cm (ID)X 25 cm (L)}. The % loading with respect to total column volume was 3.69% and the relative loading capacity with respect to traditional HPLC was about 4.

The loading capacity during the enantiomers separation using the box car injection technique was about 6.11%. This is very close to the sample loading observed in normal phase Prep HPLC where the entire exposed silica surface is available for chromatography.

Table 1, entries 5 and 6 reveal that the ASP/SSP technique described in embodiments of the invention have loading capacities in the range of 7.1% to 9.9%. The C-8 derivatized silica has a higher sample loading capacity than the C-18 derivatized silica due to the steric relief (C-8 versus C-18 chains) and consequently a higher amount of the adsorbed SSP. The higher sample loading observed with SSP aided Prep-RP-HPLC is ascribed to the increased surface area that is available as a consequence of the SSP/ASP self-assembling in to a three dimensional lattice.

The pore size of the C-18/C-8 silica is known to affect the loading capacity and the effectiveness (success) of the purification of the target compound. For example, the quality of separation of macromolecules such as proteins is better with wide-pore supports such as 300 Å or 1000 Å. A consequence of the wide pores is the decreased amount of product that could be purified in a single pass since less stationary phase is available for binding.

Smaller pore size stationary phases such as the 80 to 120 Å (Angstroms) support is preferred for smaller molecules and small peptides (5 to 15 amino acids) while wide pore silica is the preferred support for larger peptides (>25 amino acids) and proteins. Non-specific interactions between the analyte and stationary phase also influence the sample loading, purification efficiency (resolution), and output. True reversed phase interactions between the analyte and the C-18/C-8 stationary phase are reduced because of ion-exchange/ion-pair interactions with the residual silanols (which are a consequence of incomplete end capping). Also, the steric constraints between the C-18/C-8 chains influence the degree of carbon loading. The void volume of column (CVₒ) volume of a column is easily measured by measuring the elution volume of an un-retained solute. It is usually about 40% to 50% of the total column volume. A portion of this void volume is made use of for coating with the ASP/SSP. Table 1, entries 5 and 6 illustrate that greater loading is seen with the SSP coated C-8 derivatized silica in contrast to the SSP coated C-18 derivatized silica.

Control Experiments Using Various Loadings of Crude Leuprolide:

A Reveleris® (flash chromotography system) flash column containing 12 g of silica derivatized with C-18 alkyl chains was chosen, and equilibrated with about 10 column volumes (CVs) of 0.1% aqueous trifluoroacetic acid at a flow rate of 6 mL/min. Next, the column was loaded with various finite amounts of crude Leuprolide (86.4% pure by HPLC; Peptide Assay was done by Edelhoch Method) as shown in Table 2. Four parameters were studied to evaluate the chromatography performance.
1. Flow through: Amount of Leuprolide in the flow through during loading was measured. This helped ascertain whether the capacity of column during loading was exceeded.
2. Pool of fractions containing at least 95.0% Leuprolide: Several pools of fractions were made and the amount of Leuprolide was quantified using the Edelhoch Method or by quantitative HPLC assay.
3. Purest Leuprolide Fraction (Measure of Resolution): The fraction containing the highest purity of Leuprolide was determined. This was helpful in assessing the resolution of Leuprolide from its closest eluting impurities.
4. Mass balance of the entire eluent from the chromatographic run: This was measured using the Edelhoch Method. This was helpful in determining the loss of Leuprolide and similar analogues due to non-specific ionic binding to residual silanol groups present on the reversed phase column.

TABLE 2
Loading Capacity in Classical Prep RP-HPLC: Control Experiment
Control Experiments Parameters: Reveleris C-18, 12 g, 40μ, 60 \acute{Å}; Analyte: Crude
Leuprolide, 84.6% pure by modified USP Method; Flow rate was 8 mL/min; Buffer A = 0.1%
TFA in Water; Buffer B = 0.1% TFA in 50% aqueous CH3CN.

| | | Load: 100 mgs | Load: 200 mgs | | | |
|---|---|---|---|---|---|---|
| | | (Edelhoch); | (Edelhoch); | Load: 400 mgs | Load: 800 mgs | |
| | | True Wt | True Wt | (Edelhoch); | (Edelhoch); | Load: 1200 mgs |
| | | of | of | True Wt of | True Wt of | (Edelhoch); |
| | | Leuprolide | Leuprolide | Leuprolide | Leuprolide | True Wt of |
| | | (Corrected | (Corrected | (Corrected | (Corrected | Leuprolide |
| | | for 84.6% | for 84.6% | for 84.6% | for 84.6% | (Corrected for |
| | | purity): | purity): | purity): | purity): | 84.6% purity): |
| Entry | Parameter | 84.6 mgs | 169.2 mgs | 338.4 mgs | 676.8 mgs | 1015.2 mgs |
| #1 | Amount | 0 mg | 0.5 mg | 0.2 mg | 0.2 mg | 59 mg |
| | (mgs) in | | | | | |
| | Flow | | | | | |
| | Through | | | | | |
| #2 | Was there | Not | Yes. | Yes. | Yes. 80.9% | Yes. 81.0% |
| | any | determined | 72.3% | 81.36% | pure | pure |
| | Leuprolide | | pure | pure | Leuprolide | Leuprolide |
| | in 100% B | | Leuprolide | Leuprolide | | |
| | wash (% | | | | | |
| | Purity of | | | | | |
| | Leuprolide) | | | | | |
| #3 | % Highest | 97.8% | 96.5% | 96.5% | 95.2% | 95.5% |
| | Purity | | | | | |
| | (Single | | | | | |
| | Fraction) | | | | | |
| #4 | Wt of | 19.1 mgs | 30.6 mgs | 58.4 mgs | 94.8 mgs | 173.6 mgs |
| | Fractions >95% | | | | | |
| | pure | | | | | |
| | by | | | | | |
| | Edelhoch | | | | | |
| | Method | | | | | |
| #5 | % Yield of | 19.1% | 15.3% | 14.5% | 11.9% | 14.4% |
| | purification | | | | | |
| | (Edelhoch | | | | | |
| | Method) | | | | | |
| #6 | % | 22.6% | 18.1% | 17.3% | 14.0% | 17.1% |
| | Recovery | | | | | |
| | of | | | | | |
| | Leuprolide | | | | | |
| | (Edelhoch | | | | | |
| | Method) | | | | | |
| #7 | Mass | 96.3% | 88.4% | 91.6% | 92.3% | 96.5% |
| | balance | | | | | |
| | (Amt by | | | | | |
| | Edelhoch | | | | | |
| | Method/ | | | | | |
| | %) | | | | | |

Examination of Table 2 reveals that:
1. The output (% purification yield) of >95% Leuprolide ranged from 11.9% to 19.1%. Stated simply 80.9% to 88.1% of crude Leuprolide could not be purified because of non-reversed phase type of interactions between the analyte and the stationary phase!
2. The mass balance of the individual chromatography runs was in the range of 88.4% to 96.5%. This suggests the high contribution of “non-reversed type of interactions between the analyte and the stationary phase” to be the cause of poor purification performance with respect to the output of >95% pure Leuprolide.
3. The purity of the individual fractions ranged from 97.8% (when 100 mgs of crude Leuprolide was loaded) to 95.2% (when 800 mgs of crude Leuprolide was loaded). The purity was 95.5% when 1200 mgs of crude Leuprolide was loaded. This may be due to the “self-displacement” contribution.
4. A higher purification performance in terms of “Efficiency” and “Effectiveness” is possible if the residual silanol groups are effectively incapacitated towards ionic binding of analyte to the stationary phase.

Evaluation of Neutral PEG Based Surfactants:

Table 3 summarizes the performance of Triton X-100, Tween-80, and Brij-35 as ASPs. A Reveleris Silica derivatized C-18 column (12 g of stationary phase, 40 microns diameter particles, and 60 Angstroms pore size) was chosen and saturated with either 12 g of Triton X-100 or Tween-80 or Brij-35 dissolved in water.

List of Surfactants that are Useful as ASPs/SSPs

### Alkyl Glycosides

R—O—(CH₂)ₓ—CH₃ R—O—(CH₂)ₓ—CH₃

### Where R=Glucose R=Maltose

X=8, n-nonyl-β-D-glucopyranoside x=11, dodecyl-β-D-maltoside
x=7, n-octyl-β-D-glucopyranoside x=9, dodecyl-β-D-maltoside
X=6, n-heptyl-β-D-glucopyranoside x=9, decyl-β-D-maltoside
x=5, n-hexyl-β-D-glucopyranoside.

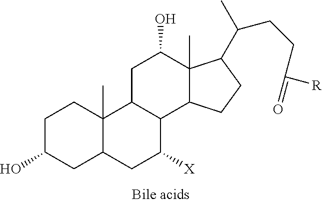

X═H, R═ONa, Sodium deoxycholate
X═H, R═NHCH₂CH₂SO₃Na, Sodium taurodeoxycholate
X═H, R═NHCH₂CH₂CO₂Na, Sodium glycodeoxycholate
X═OH, R═ONa, Sodium cholate
X═OH, R═NHCH₂CH₂SO₃Na, Sodium taurocholate
X═OH, R═NHCH₂CH₂CO₂Na, Sodium glycocholate

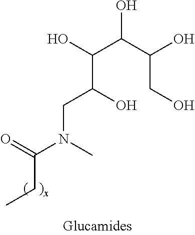

### Where

### X=8, MEGA-10

### X=7, MEGA-9,

### X=6, MEGA-8

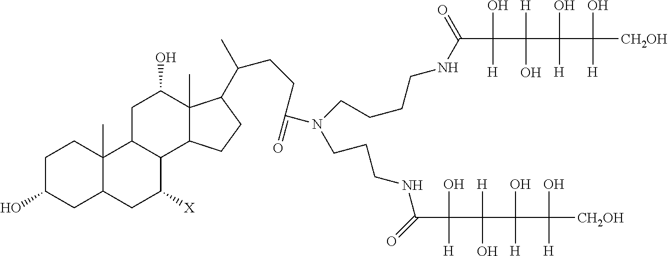

### Where,

### X═H, Deoxy Big CHAP

### X═OH, Big CHAP

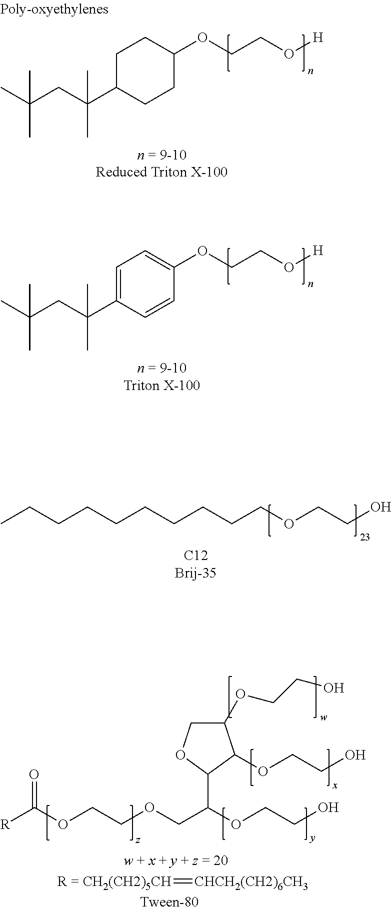

Excess un-bound detergent was removed by washing with 90% aqueous acetonitrile containing 0.1% trifluoroacetic acid. When this step was omitted premature elution of crude API was observed because the excess detergent was present at a concentration that was higher than its critical micellar concentration.

Next, the crude API (800 mgs of 81.7% Leuprolide, corrected weight of Leuprolide was 653.3 mgs) was loaded, and the column was washed with 5 CVₒ volumes of buffer A (0.1 mM Cetyltrimethylammonium bromide and 0.1 mM sodium bicarbonate in water). Analytical RP-HPLC analysis of the “flow through” eluent revealed the absence of Leuprolide.

A linear gradient of buffer B (0.1 mM Cetyltrimethylammonium bromide and 0.1 mM sodium bicarbonate in 50% aqueous acetonitrile) was started to elute the product from the column.

In contrast to the Gaussian peaks observed in traditional Prep-RP-HPLC an “M-shaped peak” is seen in SSP aided Prep-RP-HPLC. The pool of fractions containing >95% pure Leuprolide was quantitated by an HPLC assay. This served as a measure of performance/throughput of the column. The % purity of the individual fractions comprising the pool was determined by analytical RP-HPLC. The average highest purity of the individual fraction (five purification runs) was 98.84%. The average weight of the purified pool as measured by quantitative HPLC assay was 409 mgs (theoretical amount is 653.3 mgs) and the average % Leuprolide recovery was 62.6%.

The deposited ASP/SSP is removed from the reversed phase column by washing the column with 0.25 M ammonium acetate in 50% to 80% acetonitrile in water.

TABLE 3
Performance of Triton X-100, Tween-80, and Brij-35 as ASPs at 6.6% Sample
Loading

| | | C-18 Column; 12 g; 40μ; 60 \acute{Å}; Load: 800 mgs of Crude Leuprolide (Net | | | | | | | | | |
|---|---|---|---|---|---|---|---|---|---|---|---|
| | | weight by Edelhoch Method; % Purity of Crude was 81.7%; True weight | | | | | | | | | |
| | | of Leuprolide is 653.3 mgs); Buffer A = 0.1 mM | | | | | | | | | |
| | | Cetyltrimethylammonium bromide (CTA-Br) − 0.1 mM NaHCO3− Water; | | | | | | | | | |
| | | Buffer B = 0.1 mM CTA-Br− 0.1 mM NaHCO3− 50% CH3CN− 50% | | | | | | | | | |
| Entry | Parameter | Water; Flow = 6 mL/min; λ = 220 nm and 280 nm | | | | | | | | | |
| #1 | Detergent | Triton | Triton | Triton | Triton | Triton | Triton | Tween- | Tween- | Brij- | Brij- |
| | Loaded | X-100 | X- | X- | X- | X-100 | X- | 80 | 80 | 35 | 35 |
| | (12 g) | | 100 | 100 | 100 | | 100 | | | | |
| #2 | Wash: | NO | Yes | Yes | Yes | Yes | Yes | Yes | Yes | Yes | Yes |
| | 90% | | | | | | | | | | |
| | CH3CN− | | | | | | | | | | |
| | Water | | | | | | | | | | |
| | (0.1% | | | | | | | | | | |
| | TFA) | | | | | | | | | | |
| #3 | Was there | NA | NO | NO | NO | NO | NO | NO | NO | NO | NO |
| | any | | | | | | | | | | |
| | Leuprolide | | | | | | | | | | |
| | present in | | | | | | | | | | |
| | flow | | | | | | | | | | |
| | through | | | | | | | | | | |
| | (FT) or | | | | | | | | | | |
| | 100% B | | | | | | | | | | |
| | wash | | | | | | | | | | |
| #4 | Pool of | Compound | 95.5% | 95.8% | 95.5% | 96.0% | 96.3% | 95.5% | 95.2% | 96.0% | 95.6% |
| | Fractions | eluted | | | | | | | | | |
| | with >95% | in the | | | | | | | | | |
| | Purity; | void | | | | | | | | | |
| | Mod. EP | volume | | | | | | | | | |
| | Gradient | | | | | | | | | | |
| | Method; | | | | | | | | | | |
| | % Purity | | | | | | | | | | |
| #5 | Single | NA | 98.7% | 99.3% | 99.2% | 98.5% | 98.5% | 96.6% | 95.9% | 98.5% | 97.8% |
| | fraction | | | | | | | | | | |
| | with | | | | | | | | | | |
| | highest | | | | | | | | | | |
| | prity; % | | | | | | | | | | |
| #6 | Yield (by | NA | 381.2 mgs; | 413.3 mgs; | 412.9 mgs; | 420 mgs; | 417.3 | 321.6 mgs; | 365.5 | 406.0 mgs; | 382.7 |
| | Quantitative | | 47.6% | 51.7% | 51.6% | 52.5% | mgs; | 40.2% | mgs; | 50.8% | mgs; |
| | HPLC)- | | | | | | 52.2% | | 45.7% | | 47.8% |
| | Weight | | | | | | | | | | |
| | (mgs); % | | | | | | | | | | |
| | Yield | | | | | | | | | | |
| #7 | % | NA | 381.2 mgs; | 413.3 mgs; | 412.9 mgs; | 420 mgs; | 417.3 | 321.6 mgs; | 365.5 | 406.0 mgs; | 382.7 |
| | Recovery | | 58.3% | 63.3% | 63.2% | 64.3% | mgs; | 49.2% | mgs; | 62.1% | mgs; |
| | (Wt of | | | | | | 63.9% | | 55.9% | | 58.6% |
| | pure | | | | | | | | | | |
| | Leuprolide | | | | | | | | | | |
| | by quan | | | | | | | | | | |
| | HPLC/ | | | | | | | | | | |
| | 653.3 mgs | | | | | | | | | | |
| | for a 800 mgs | | | | | | | | | | |
| | feed) | | | | | | | | | | |

Similar experiments performed with Tween-80 (average of 2 runs) furnished the following data: (1) Fraction with the highest average individual purity was 96.25%; (2) Average weight of the >95% purified pool was 343.6 mgs (theoretical amount is 653.3 mgs) and the average % Leuprolide recovery yield was 42.95%.

Similar experiments performed with Brij-35 (average of 2 runs) furnished the following data: (1) Fraction with the highest average individual purity was 98.15%; (2) Average weight of the >95% purified pool was 394.4 mgs (theoretical amount is 653.3 mgs) and the average % Leuprolide recovery yield was 60.35%.

The above results suggest that Triton X-100 was the optimum of the three SSPs evaluated for the purification of Leuprolide.

The next series of experiments examined the influence of varying pore sizes and diameter of C-18 derivatized silica particles on the Prep-HPLC yield, and are summarized in Table 4. Two Reveleris (Column Parameters: 12 g of C-18, 40μ, 60 Angstrom, and Column Parameters: 12 g of C-18, 20μ, 150 Angstrom) and one Peerless Basic C-18 (packed in house, Column Parameters: About 12 g of C-18, 10μ, 100 Angstrom) C-18 derivatized silica columns were used. The columns were saturated with 12 g of Triton X-100 dissolved in water. Excess un-bound detergent was removed by washing with 90% aqueous acetonitrile containing 0.1% trifluoroacetic acid.

The column was equilibrated with 5 CVₒ volumes of buffer A (0.1% aqueous phosphoric acid). Next, the crude API (800 mgs of 81.7% Leuprolide, corrected weight of Leuprolide was 653.3 mgs) was loaded, and the column was washed with 5 CVₒ volumes of buffer A. Analytical RP-HPLC analysis of the “flow through” eluent revealed the absence of Leuprolide.

A linear gradient of buffer B (0.1% aqueous phosphoric acid in 50% aqueous acetonitrile) was started to elute the product from the column.

The pool of fractions containing >95% pure Leuprolide was quantitated by HPLC assay. This served as a measure of performance/throughput of the column. The % purity of the individual fractions comprising the pool was determined by analytical RP-HPLC.

The average highest purity of the individual fraction (two purification runs performed with 40μ support) was 99.3%. The average weight of the purified pool as measured by quantitative HPLC assay was 467 mgs (theoretical amount is 653.3 mgs) and the average % Leuprolide recovery was 71.5%.

TABLE 4
Use of Triton X-100 as ASP on 40 micron, 20 micron, and 10 micron columns in
phosphoric acid buffers

| | | C-18 Column; 12 g; 20μ/0r 40μ; 60 \acute{Å} or 150 \acute{Å}; Load: 800 mgs of | | | | | |
|---|---|---|---|---|---|---|---|
| | | Crude Leuprolide (Net weight by Edelhoch Method); Buffer A = 0.1% | | | | | |
| | | H3PO4− Water; Buffer B = 0.1% H3PO4 in 50% CH3CN− 50% Water; | | | | | |
| Entry | Parameter | Flow = 6 mL/min; λ = 220 nm and 280 nm | | | | | |
| #1 | Detergent | Triton | Triton | Triton X- | Triton X- | Triton X- | Triton X- |
| | Load (12 g) | X-100; | X-100; | 100; | 100; | 100; | 100; |
| | | Reveleris | Reveleris | Reveleris | Peerless | Peerless | Peerless |
| | | C-18 | C-18 | C-18 | Basic C- | Basic C- | Basic C-18 |
| | | Column: | Column: | Column: | 18 | 18 | Column: 10μ; |
| | | 40μ; 60 \acute{Å} | 40μ; 60 \acute{Å} | 20μ; 150 \acute{Å} | Column: | Column: | 100 \acute{Å} |
| | | | | | 10μ; 100 \acute{Å} | 10μ; 100 \acute{Å} | (Packed in- |
| | | | | | (Packed | (Packed | house-2 |
| | | | | | in-house) | in- | trial); 1% |
| | | | | | | house); | H3PO4 was |
| | | | | | | 1% | used instead |
| | | | | | | H3PO4 | of 0.1%. |
| | | | | | | was used | |
| | | | | | | instead of | |
| | | | | | | 0.1%. | |
| #2 | Wash: 90% | Yes | Yes | Yes | Yes | Yes | Yes |
| | CH3CN− | | | | | | |
| | Water | | | | | | |
| | (0.1% TFA) | | | | | | |
| #3 | Was there | NO | NO | NO | NO | NO | NO |
| | any | | | | | | |
| | Leuprolide | | | | | | |
| | present in | | | | | | |
| | flow | | | | | | |
| | through | | | | | | |
| | (FT) or | | | | | | |
| | 100% B | | | | | | |
| | wash | | | | | | |
| #4 | Fractions #s | 97.3% | 97.3% | 97.9% | 96.4% | 96.6% | 95.6% |
| | (>95% | | | | | | |
| | Purity; | | | | | | |
| | Mod. EP | | | | | | |
| | Gradient | | | | | | |
| | Method); % | | | | | | |
| | Purity | | | | | | |
| #5 | Single | 99.3% | 99.3% | 99.2% | 98.9% | 98.6% | 98.1% |
| | fraction | | | | | | |
| | with highest | | | | | | |
| | purity; % | | | | | | |
| #6 | Yield (by | 466.9 mgs; | 466.9 mgs; | 528.0 mgs; | 504.2 mgs; | 512.3 mgs; | 497.3 mgs; |
| | Quantitative | 58.4% | 58.4% | 66.0% | 63.0% | 64.0% | 62.2% |
| | HPLC)- | | | | | | |
| | Weight | | | | | | |
| | (mgs); % | | | | | | |
| | Yield (Wt | | | | | | |
| | in mgs/800 mgs) | | | | | | |
| #7 | % Recovery | 71.5% | 71.5% | 80.8% | 77.2% | 78.9% | 76.6% |
| | (Wt of pure | | | | | | |
| | Leuprolide | | | | | | |
| | by quan | | | | | | |
| | HPLC/ | | | | | | |
| | 653.3 mgs | | | | | | |
| | for a 800 mgs | | | | | | |
| | feed) | | | | | | |
| | {corrected | | | | | | |
| | for % | | | | | | |
| | purity} | | | | | | |

The average highest purity of the individual fraction (one purification run performed with the Reveleris C-18 20μ support) was 99.3%. The weight of the purified pool as measured by quantitative HPLC assay was 528 mgs (theoretical amount is 653.3 mgs) and the % Leuprolide recovery was 80.8%.

The results were similar to the Reveleris C-18 20μ support to a previously used Peerles Basic C-18 10μ support column.

Table 5 reveals that using increasing concentrations of triethylammonium phosphate caused a decrease in purification yield.

TABLE 5
Use of Triton X-100 ASP in triethylammonium phosphate buffers

| | | C-18 Column; 12 g; 40μ; 60 \acute{Å}; Load: 800 mgs of Crude | |
|---|---|---|---|
| | | Leuprolide (Net weight by Edelhoch Method; True wt of | |
| | | Leuprolide is 653.3 mgs); Buffer A = x mM TEAP (pH 3)- | |
| | | Water; Buffer B = x mM TEAP (pH 3) in 50% CH3CN-50% | |
| Entry | Parameter | Water; Flow = 6 mL/min; λ = 220 nm and 280 nm | |
| #1 | Detergent Load (12 g) | Triton X-100; C-18 Column: | Triton X-100; C-18 Column: |
| | | 40μ; 60 \acute{Å}; x = 25 mM | 40μ; 60 \acute{Å}; x = 150 mM |
| #2 | Wash: 90% CH3CN- | Yes | Yes |
| | Water (0.1% TFA) | | |
| #3 | Was there any | NO | NO |
| | Leuprolide present in | | |
| | flow through (FT) or | | |
| | 100% B wash | | |
| #4 | Fractions #s (>95% | 11 to 31; 97.0% | 8 to 15; 97.1% |
| | Purity; Mod. EP | | |
| | Gradient Method); % | | |
| | Purity | | |
| #5 | Single fraction with | 26; 98.6% | 13; 98.3% |
| | highest purity; % | | |
| #6 | Yield (by Quantitative | 314.5 mgs; 39.3% | 280 mgs; 35.0% |
| | HPLC)-Weight | | |
| | (mgs); % Yield | | |
| #7 | % Recovery (Wt of | 48.1% | 42.9% |
| | pure Leuprolide by | | |
| | quan HPLC/653.3 mgs | | |
| | for a 800 mgs | | |
| | feed) | | |

A Reveleris® (Column Parameters: 12 g of C-18, 40μ, 60 Angstrom) was saturated with 12 g of Triton X-100 dissolved in water. Excess un-bound detergent was removed by washing with 90% aqueous acetonitrile containing 0.1% trifluoroacetic acid.

The column was equilibrated with 5 CVₒ volumes of buffer A (25 mM aqueous triethylammonium phosphate, pH 3). Next, the crude API (800 mgs of 81.7% Leuprolide, corrected weight of Leuprolide was 653.3 mgs) was loaded, and the column was washed with 5 CVₒ volumes of buffer A. Analytical RP-HPLC analysis of the “flow through” eluent revealed the absence of Leuprolide.

A linear gradient of buffer B {25 mM aqueous triethylammonium phosphate (pH 3) in 50% aqueous acetonitrile} was started to elute the product from the column.

The pool of fractions containing >95% pure Leuprolide was quantitated by HPLC assay. This served as a measure of performance/throughput of the column. The % purity of the individual fractions comprising the pool was determined by analytical RP-HPLC. The highest purity of the individual fraction was 98.6%. The weight of the purified pool as measured by quantitative HPLC assay was 314.5 mgs (theoretical amount is 653.3 mgs) and the % Leuprolide recovery was 48.1%.

The subsequent experiment was done with a higher concentration of triethylammonium phosphate, namely 150 mM aqueous triethylammonium phosphate at pH 3. The column was equilibrated with 5 CVₒ volumes of buffer A (150 mM aqueous triethylammonium phosphate, pH 3). Next, the crude API (800 mgs of 81.7% Leuprolide, corrected weight of Leuprolide was 653.3 mgs) was loaded, and the column was washed with 5 CVₒ volumes of buffer A. Analytical RP-HPLC analysis of the “flow through” eluent revealed the absence of Leuprolide.

A linear gradient of buffer B {150 mM aqueous triethylammonium phosphate (pH 3) in 50% aqueous acetonitrile} was started to elute the product from the column.

The pool of fractions containing >95% pure Leuprolide was quantitated by HPLC assay. This served as a measure of performance/throughput of the column. The % purity of the individual fractions comprising the pool was determined by analytical RP-HPLC.

The highest purity of the individual fraction was 98.3%. The weight of the purified pool as measured by quantitative HPLC assay was 280 mgs (theoretical amount is 653.3 mgs) and the % Leuprolide recovery was 42.9%. The lower yield observed with triethylammonium phosphate buffers (43% to 48%) in contrast to phosphoric acid buffers (71% to 80%) reveals that SSP-bound to silanols is partially lost.

As described above, conventional RP-HPLC hardware systems may be used for the separation, and the term “configuring a chromatographic system” refers to setting up a column or system of column, pump and detector as well known in the art.

The term “saturating the chromatographic stationary phase” refers to passing the surfactant in a solution over the stationary phase in a particular concentration, thereby preparing the surrogate stationary phase.

Preferable methods of embodiments of the present invention are mentioned below: Illustrative Method for purifying organic molecules including peptides using surfactants as surrogate stationary phase:

It is emphasized here that the following is an example merely described for illustrative purposes and is not intended to restrict the scope and utility of SSP aided Prep-RP-HPLC technique. The C-18 column used in these studies contained 12 g of C-18 derivatized silica (10μ, 20μ, or 40μ diameter particles, 60 Å, 100 Å or 150 Å pore sizes). The C-18 derivatized silica reversed phase column was equilibrated with an aqueous solution of the surfactant (such as Triton X-100, Tween-80, or Brij-35 or any neutral surfactant containing hydrogen bond acceptor sites). The weight of surfactant was in the range of 1% to 100% of the weight of the stationary phase. To ensure maximum deposition of the additional (surrogate) stationary phase 12 g of the surfactant dissolved in 500 mL of water was used. The column was then washed with 90% aqueous acetonitrile containing 0.1% trifluoroacetic acid to remove the un-bound surfactant.

Next, the column is equilibrated with starting mobile phase {10 column volumes (CVs), for example, 0.1% aqueous phosphoric acid}, and the crude product was loaded. A linear gradient of Buffer B (for example, 0.1% phosphoric acid in 50% aqueous acetonitrile) was run. When the product of interest (API) is about to elute, a gradient hold may be applied until all the API has eluted from the column (please see FIG. 2). Alternately if it is desired to elute the product in a concentrated form the gradient may be allowed to run its course. The fractions containing >95% pure API product are combined. The organic volatiles are removed under reduced pressure. The aqueous residue is passed through a C-18 column (using aqueous acetic acid and acetonitrile) to exchange the counter phosphate ion to the desired counter ion (for example, acetate ion).

Embodiments of the invention are applicable for any size column or HPLC equipment used for chromatography applications in the pharmaceutical and fine chemical industries.

Some aspects and embodiments of this disclosure are described in the examples below, which are provided only for the purpose of illustration and are not intended to limit the scope of the disclosure in any manner.

### EXAMPLES

### Example-1

### Prep-RP-HPLC of Leuprolide Acetate Using Triton X-100 as Additional Stationary Phase and Aqueous Phosphoric Acid Buffers

The C-18 reversed phase column (Reveleris C-18, 12 g, 40μ, 60 Å pore size) was saturated with Triton X-100 (12 g dissolved in 500 mL water). The excess un-bound surfactant was washed with 90% aqueous acetonitrile containing 0.1% trifluoroacetic acid to remove the un-bound surfactant. Next, the column was equilibrated with 5 column volumes (CVs) of 0.1% aqueous phosphoric acid (Buffer A). Crude Leuprolide (800 mgs, net weight by Edelhoch method) dissolved in buffer A was loaded on to the column. The column was washed with 5 CVs of Buffer A. Analytical RP-HPLC analysis of the “flow through” eluent revealed the absence of Leuprolide. When this preceding step was omitted premature elution of crude API was observed because the excess surfactant was present at a concentration that was higher than its critical micellar concentration. Next, the gradient elution process was started. Buffer B was 0.1% phosphoric acid in 50% aqueous acetonitrile. A linear gradient of 0% B to 100% Buffer B over 60 min. was used for elution. A gradient hold was applied until all the API had eluted from the column. The fractions containing >95% pure API product were combined. The Prep-HPLC profile is shown in FIG. 2. The experiment was performed in duplicate.

The pool of fractions containing >95% pure Leuprolide was quantitated by HPLC assay. The average highest purity of the individual fraction (two purification runs) was 99.3%. The average weight of the purified pool as measured by quantitative HPLC assay was 466.9 mgs (theoretical amount is 653.3 mgs) and the average % Leuprolide recovery was 71.5%.

### Example 2

### Prep-RP-HPLC of Leuprolide Acetate Using Triton X-100 as Additional Stationary Phases and 0.1 mM Cetyltrimethylammonium Bromide Buffers

Reveleris® Silica derivatized C-18 column (12 g of stationary phase, 40 microns diameter particles, and 60 Angstroms pore size) was chosen and saturated with 12 g of Triton X-100 dissolved in water.

Excess un-bound surfactant was removed by washing with 90% aqueous acetonitrile containing 0.1% trifluoroacetic acid. When this step was omitted premature elution of crude API was observed because the excess surfactant was present at a concentration that was higher than its critical micellar concentration.

Next, the crude API (800 mgs of 81.7% Leuprolide, corrected weight of Leuprolide was 653.3 mgs) was loaded, and the column was washed with 5 CVs of buffer A (0.1 mM Cetyltrimethylammonium bromide and 0.1 mM sodium bicarbonate in water). Analytical RP-HPLC analysis of the “flow through” eluent revealed the absence of Leuprolide.

A linear gradient of buffer B (0.1 mM Cetyltrimethylammonium bromide and 0.1 mM sodium bicarbonate in 50% aqueous acetonitrile) was started to elute the product from the column.

The pool of fractions containing >95% pure Leuprolide was quantitated by HPLC assay. This served as a measure of performance/throughput of the column. The % purity of the individual fractions comprising the pool was determined by analytical RP-HPLC. The average highest purity of the individual fraction (five purification runs with Triton X-100) was 98.8%. The average weight of the purified pool as measured by quantitative HPLC assay was 408.9 mgs (theoretical amount is 653.3 mgs) and the average % Leuprolide recovery was 62.6%.

### Example 3

### Prep-RP-HPLC of Leuprolide Acetate Using Tween 80 as Additional Stationary Phases and 0.1 mM Cetyltrimethylammonium Bromide Buffers

Reveleris® Silica derivatized C-18 column (12 g of stationary phase, 40 microns diameter particles, and 60 Angstroms pore size) was chosen and saturated with 12 g of Tween-80 dissolved in water.

Excess un-bound surfactant was removed by washing with 90% aqueous acetonitrile containing 0.1% trifluoroacetic acid. When this step was omitted premature elution of crude API was observed because the excess surfactant was present at a concentration that was higher than its critical micellar concentration.

Next, the crude API (800 mgs of 81.7% Leuprolide, corrected weight of Leuprolide was 653.3 mgs) was loaded, and the column was washed with 5 CVs of buffer A (0.1 mM Cetyltrimethylammonium bromide and 0.1 mM sodium bicarbonate in water). Analytical RP-HPLC analysis of the “flow through” eluent revealed the absence of Leuprolide.

A linear gradient of buffer B (0.1 mM Cetyltrimethylammonium bromide and 0.1 mM sodium bicarbonate in 50% aqueous acetonitrile) was started to elute the product from the column.

The pool of fractions containing >95% pure Leuprolide was quantitated by HPLC assay. This served as a measure of performance/throughput of the column. The % purity of the individual fractions comprising the pool was determined by analytical RP-HPLC.

The experiment was performed in duplicate, and it furnished the following data: (1) Fraction with the highest average individual purity was 96.3%; (2) Average weight of the >95% purified pool was 343.6 mgs (theoretical amount is 653.3 mgs) and the average % Leuprolide recovery yield was 52.6%.

### Example 4

### Prep-RP-HPLC of Leuprolide Acetate Using Brij-35 as Additional Stationary Phases and 0.1 mM Cetyltrimethylammonium Bromide Buffers

Reveleris Silica derivatized C-18 column (12 g of stationary phase, 40 microns diameter particles, and 60 Angstroms pore size) was chosen and saturated with 12 g of Brij-35 dissolved in water.

Excess un-bound surfactant was removed by washing with 90% aqueous acetonitrile containing 0.1% trifluoroacetic acid. When this step was omitted premature elution of crude API was observed because the excess surfactant was present at a concentration that was higher than its critical micellar concentration.

Next, the crude API (800 mgs of 81.7% Leuprolide, corrected weight of Leuprolide was 653.3 mgs) was loaded, and the column was washed with 5 CVs of buffer A (0.1 mM Cetyltrimethylammonium bromide and 0.1 mM sodium bicarbonate in water). Analytical RP-HPLC analysis of the “flow through” eluent revealed the absence of Leuprolide.

A linear gradient of buffer B (0.1 mM Cetyltrimethylammonium bromide and 0.1 mM sodium bicarbonate in 50% aqueous acetonitrile) was started to elute the product from the column.

The pool of fractions containing >95% pure Leuprolide was quantitated by HPLC assay. This served as a measure of performance/throughput of the column. The % purity of the individual fractions comprising the pool was determined by analytical RP-HPLC.

The experiment was performed in duplicate and it furnished the following data: (1) Fraction with the highest average individual purity was 98.2%; (2) Average weight of the >95% purified pool was 394.4 mgs (theoretical amount is 653.3 mgs) and the average % Leuprolide recovery yield was 60.4%.

1. A method of purification of organic compounds including: peptides with increased sample loading capacity of reverse phase column in Preparative Reversed Phase High Performance Liquid Chromatography (Prep-RP-HPLC) using one of a surrogate stationary phase and an additional stationary phase in conjunction with one of a C-18 and a C-8 derivatized silica stationary phase.

2. The method as claimed in claim 1, wherein the preparative loading capacity of one of the C-18 and the C-8 reverse phase column is increased by one of coating and binding the one of the C-18 and the C-8 reverse phase column with one of the surrogate stationary phase and the additional stationary phase.

3. The method as claimed in claim 2, wherein one of the surrogate stationary phase and the additional stationary phase is a neutral surfactant or pegylated surfactant.

4. The method as claimed in claim 3, wherein one of the surrogate stationary phase and the additional stationary phase surfactants are selected from alkyl glycoside, bile acids, glucamides and poly-oxyethylenes.

5. The method as claimed in claim 4, wherein the poly-oxyethylenes are selected from Triton X-100, Tween-80 and Brij-35.

6. The method as claimed in claim 5, wherein the surfactant is Triton X-100.

7. The method as claimed in claim 4, wherein alkyl glycosides are selected from the compounds have the formula of R—O—(CH₂)ₓ—CH₃,
wherein,
When R=Glucose When R=Maltose
X=8, n-nonyl-β-D-glucopyranoside x=11, dodecyl-β-D-maltoside
x=7, n-octyl-β-D-glucopyranoside x=9, dodecyl-β-D-maltoside
X=6, n-heptyl-β-D-glucopyranoside x=9, decyl-β-D-maltoside
x=5, n-hexyl-β-D-glucopyranoside.

8. The method as claimed in claim 4, wherein the bile acids are selected from the compounds having formula: 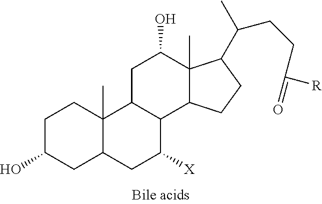
wherein,
X═H, R═ONa, Sodium deoxycholate
X═H, R═NHCH₂CH₂SO₃Na, Sodium taurodeoxycholate
X═H, R═NHCH₂CH₂CO₂Na, Sodium glycodeoxycholate
X═OH, R═ONa, Sodium cholate
X═OH, R═NHCH₂CH₂SO₃Na, Sodium taurocholate
X═OH, R═NHCH₂CH₂CO₂Na, Sodium glycocholate

9. The method as claimed in claim 4, wherein glucamides are selected from the compounds having formula: 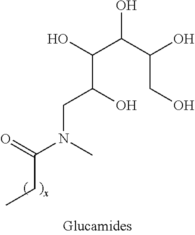
wherein,
X=8, MEGA-10
X=7, MEGA-9,
X=6, MEGA-8
or, compound of the formula: 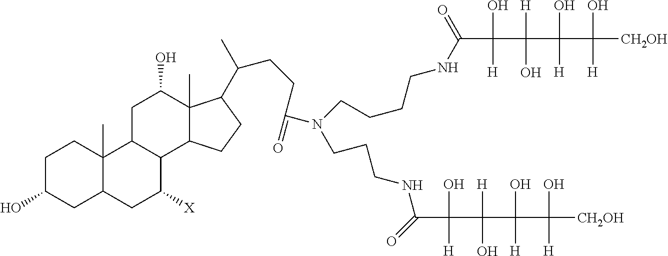
wherein,
X═H, Deoxy Big CHAP
X═OH, Big CHAP

10. A method of purifying a multi-component sample of organic compounds including peptides by Preparative Reversed Phase High Performance Liquid Chromatography (Prep-RP-HPLC) comprising the steps of:
(a) configuring a chromatographic system having a hydrophobic stationary phase;
(b) saturating the chromatographic stationary phase with one of a surrogate stationary phase and additional stationary phase surfactants selected from alkyl glycoside, bile acids, glucamides and poly-oxyethylenes;
(c) washing the column to remove excess un-bound surfactant employing a mixture of organic solvents and water;
(d) equilibrating the column with the starting mobile phase;
(e) applying a multicomponent sample to one end of the chromatographic bed comprising stationary phase coated with the surfactants; and
(f) eluting the multicomponent sample using a linear gradient of buffers A & B, wherein the buffer A is 0.1 mM Cetyltrimethylammonium bromide and 0.1 mM sodium bicarbonate in water and buffer B is 0.1 mM Cetyltrimethylammonium bromide and 0.1 mM sodium bicarbonate in 50% aqueous acetonitrile;
(g) eluting the multicomponent sample using a linear gradient of buffers A & B, wherein the buffer A is 0.1% phosphoric acid in water and buffer B is 0.1% phosphoric acid in 50% aqueous acetonitrile; or
(h) eluting the multicomponent sample using a linear gradient of buffers A & B, wherein the buffer A is 1% phosphoric acid in water and buffer B is 1% phosphoric acid in 50% aqueous acetonitrile; or
(i) eluting the multicomponent sample using a linear gradient of buffers A & B, wherein the buffer A is 25 mM to 150 mM triethylammonium phosphate (pH 3) in water and buffer B is 25 mM to 150 mM triethylammonium phosphate (pH 3) in 50% aqueous acetonitrile; and
(j) recovering the desired component of the sample.

11. The method as claimed in claim 10, wherein the hydrophobic stationary phase in step (a) is C-8 or C-18 alkyl chain derivatized silica.

12. The method as claimed in claim 10, wherein the surfactants in step (b) is selected from Triton X-100, Tween-80, and Brij-35.

13. The method as claimed in claim 10, wherein the washing of the column in step (c) to remove the un-bound surfactant comprises washing the column with aqueous acetonitrile more preferably 90% aqueous acetonitrile containing 0.1% trifluoroacetic acid.

14. The method as claimed in 10, wherein the equilibration comprises equilibrating the column with the starting mobile phase more preferably 0.1% to 1% aqueous phosphoric acid, 0.1% TFA in water, and 25 to 150 mM triethylammonium phosphate in water.

15. A method of purification of organic compounds including peptides with increased sample loading capacity of reverse phase column in Preparative Reversed Phase High Performance Liquid Chromatography (Prep-RP-HPLC) using a PEG based detergent/surfactant which has the following structure as one of ASP and SSP in conjunction with one of C-18 and C-8 derivatized silica or other supports as the stationary phase: 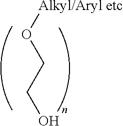
wherein one of alkyl, aryl, cyclic, and aromatic are selected independently from the group comprising straight or branched alkyl, cyclic hydrocarbons, aromatic group, alkyl substituted aromatic group, aryl substituted alkyl groups; and “n” is the number of ethylene-oxide residues from 1 to 20.

16. The method as claimed in claim 1, wherein the increase in sample loading capacity occurs when the surrogate stationary phase bound to C-18 derivatized silica is mobile (as observed with lower carbon based surfactants where concurrent binding and leaching from the stationary phase are seen), and also when it is tightly to permanently bound to the one of the C-18 and the C-8 reversed stationary phase.

17. The method as claimed in claim 16, wherein one of the C-18 and the C-8 reversed stationary phase is selected from Triton X-100, Brij-35 and Tween-80.

18. The method as claimed in claim 1 further comprising removing the one of a surrogate stationary phase and the additional stationary phase coating from one of the C-18 and the C-8 derivatized silica support by washing the column with a buffer capable of H-bonding with the residual silanols and having sufficient concentration of organic modifier.

19. The method as claimed in claim 18, wherein the organic modifier is 0.25M to 0.5M ammonium acetate in 50% to 90% aqueous acetonitrile.
